(19) United States
(12) Patent Application Publication
(10) Pub. No.: US 2011/0257460 A1
(43) Pub. Date: Oct. 20, 2011

(54) CAPTURING AND DELIVERING MOLECULES IN A MAMMAL

(76) Inventors: David R. Holmes, Rochester, MN (US); Gurpreet S. Sandhu, Rochester, MN (US); Robert D. Simari, Rochester, MN (US); Nicole P. Sandhu, Rochester, MN (US); David R. Holmes, III, Rochester, MN (US); Jonathan Harburn, Tyne & Wear (GB)

(21) Appl. No.: 13/129,298

(22) PCT Filed: Nov. 13, 2009
(86) PCT No.: PCT/US2009/064419

§ 371 Date: May 13, 2011

Related U.S. Application Data
(60) Provisional application No. 61/114,131, filed on Nov. 13, 2008.

(51) Int. Cl. A61M 37/00; A61P 35/00; A61K 38/02

(52) U.S. Cl. 600/9; 514/1.1

(57) ABSTRACT

This document relates to molecularly imprinted polymers for use in mammals. For example, methods and materials for capturing a molecule in the body of a mammal using a molecularly imprinted polymer are provided. In some cases methods for delivering a molecule using a molecularly imprinted polymer into the body of a mammal are provided. The methods provided can be used to target any molecule in the body of a mammal.

### CROSS-REFERENCE TO RELATED APPLICATIONS

This application claims the benefit of U.S. Provisional Application Ser. No. 61/114,131, filed Nov. 13, 2008. The disclosures of the prior application is considered part of (and is incorporated by reference in) the disclosure of this application.

### BACKGROUND

1. Technical Field

This document relates to molecularly imprinted polymers for use in mammals. For example, this document provides methods for using molecularly imprinted polymers (MIPs) to capture molecules in the body of a mammal, as well as, methods for using MIPs to release molecules into the body of a mammal.

2. Background Information

A molecularly imprinted polymer (MIP) is a polymer capable of molecular recognition. A MIP can be formed by cross-linking a complex of a template molecule and functional monomers. After cross-linking, a template molecule can be removed to produce a cavity in a polymer that can recognize a target molecule. A functional monomer can have a functional group capable of interacting with a corresponding site on a template molecule. Various functional groups can make the monomer/template molecule complex stable and can improve the selectivity of a MIP for a target molecule. For example, the strength of an interaction between a template molecule and a monomer can determine the affinity and selectivity of a MIP's recognition site for a target molecule.

### SUMMARY

This document relates to molecularly imprinted polymers for use in mammals. For example, this document provides methods for using molecularly imprinted polymers (MIPs) to capture molecules in the body of a mammal, as well as, methods for using MIPs to release molecules into the body of a mammal. For example, capturing a molecule (e.g., a toxin or a disease marker) present in a mammal can allow treatment of intoxication, or diagnosis of a disease state (e.g., Alzheimer's disease). For example, releasing a molecule (e.g., a drug) into the body of a mammal can allow controlled delivery of a drug for treatment of a disease (e.g., a carcinoma).

In general, an aspect of this document features a method for capturing a molecule present within a mammal. The method comprises, or consists essential of, administering, to the mammal, a composition comprising a molecularly imprinted polymer comprising a recognition site for the molecule under conditions wherein the molecule binds to the molecularly imprinted polymer to form a molecularly imprinted molecule-molecule complex, thereby capturing the molecule. The molecule can comprise a disease marker. The disease marker can be associated with a neurodegenerative disease. The neurodegenerative disease can be Alzheimer's disease. The molecule can comprise a toxin. The toxin can be a pesticide. The toxin can be a drug. The drug can be selected from the group consisting of anti-coagulants, sedatives, and narcotics. The sedative can be a benzodiazepine. The narcotic can be an opioid. The toxin can be a heavy metal. The heavy metal can be lead. The mammal can be a human. The molecularly imprinted polymer can comprise a magnetic particle. The molecularly imprinted polymer can comprises a contrast agent. The molecularly imprinted polymer can comprises a polypeptide. The polypeptide can be a metalloproteinase. The polypeptide can be a fluorescent polypeptide. The excitation of the fluorescent polypeptide can comprise red or far-red excitation. The administrating step can comprise intravenous, subcutaneous, or oral administration. The molecule can bind to the molecularly imprinted polymer with an affinity of at least 10⁴ mol⁻¹. The method can be used to treat intoxication in a mammal. The method can include removing the complex. The removing step can comprise use of a magnetic field. The method can be used to identify the mammal as having a disease.

In another embodiment, this document features, a method for releasing a molecule within a mammal. The method comprises, or consists essential of, administering, to the mammal, a composition comprising a molecularly imprinted polymer and the molecule, wherein the molecule is releasably bound to the molecularly imprinted polymer at a recognition site for binding the molecule to form a molecularly imprinted polymer-molecule complex, under conditions wherein the molecule dissociates from the complex, thereby releasing the molecule. The molecule can be selected from the group consisting of anti-thrombolytic drugs, anti-neoplastic drugs, and anti-microbial drugs. The mammal can be a human. The molecularly imprinted polymer can comprise one or more additional recognition sites. One of the one or more additional recognition sites can be capable of binding an organ, tissue, or cell. The cell can be a cancer cell, a microbial cell, or a cell comprising a virus. The molecularly imprinted polymer-molecule complex can have a dissociation constant of at least 10⁴ mol⁻¹. The dissociation condition can comprise a stimulus. The stimulus can comprise a chemical, electrical, magnetic, or mechanical stimulus.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the description below. Other features, objects, and advantages of the invention will be apparent from the description and from the claims.

### DETAILED DESCRIPTION

This document relates to molecularly imprinted polymers for use in mammals. For example, this document provides methods for using molecularly imprinted polymers (MIPs) to capture molecules in the body of a mammal, as well as, methods for using MIPs to release molecules into the body of a mammal. For example, the methods provided herein can include administering a composition including a molecularly imprinted polymer (MIP) to a mammal.

As described herein, a “MIP” can be any polymer that is capable of molecular recognition and suitable for use in a mammal. For example, a MIP can be a polymer that is capable of binding a target molecule at a recognition site. In some cases, a recognition site of a MIP can be formed within the polymer by cross-linking functional monomers around a template molecule in a three-dimensional network of connected molecules. For example, a recognition site can be a cavity within a polymer, exposed after a template molecule is removed. In some cases, a recognition site can rebind a template molecule, or a target molecule with topology similar to the topology of a template molecule.

A MIP for use with a method as described herein can be formed using any appropriate technique for constructing a recognition site in a polymer (see, e.g., U.S. Pat. No. 6,316,235, U.S. Pat. No. 5,587273, U.S. Pat. No. 5,821,311, U.S. Pat. No. 5,872,198, U.S. Pat. No. 5,959,050, U.S. Pat. No. 6,310,110, U.S. Pat. No. 6,582,971, U.S. Pat. No. 6,780,323, U.S. Pat. No. 6,960,645, U.S. Pat. No. 7,001,963 and U.S. Pat. App. Pub. No. US 2007/0190084). Suitable techniques can include a covalent approach, a non-covalent approach, a semi-covalent approach, or a metal ion mediated approach. For example, a covalent approach can be used when the target molecule has functional groups capable of forming covalent bonds, such as alcohols (diols), aldehydes, ketones, amines and carboxylic acids. In some cases, a MIP as described herein can be prepared via non-covalent binding, including ionic bonds, hydrophobic interactions, hydrogen bonds, Van der Waals forces, and dipole-dipole bonds. For example, a MIP can be formed via non-covalent bonding of functional monomer to target molecule with a stoichiometry of 2:1 or 1:1.

A MIP can be constructed from any appropriate monomer that can be cross-linked to form a polymer suitable for administration to a mammal. Suitable polymers can include, without limitation, biodegradable polymers, hydrogels, hydrophilic polymers, hydrophobic polymers, natural polymers, oligonucleotide polymers and copolymers, and polyethylene glycol (PEG)-based polymers. In some cases, a functional monomer for preparing a MIP can be a commercially available functional monomer (e.g., methacrylic acid and 4-vinylbenzoic acid), a custom monomer (e.g., a polymerizable cyclodextrin, crown ethers, 2-6-bis-acrylamidopyridine, 2-acrylamido-pyridine, a polymerizable derivative of adenine and methacrylamide-based functional monomers), a combination of functional monomers (e.g., 2-vinylpyridine, 4-vinylpyridine, and acrylamide can be combined with methacrylic acid, acrylamide can be combined with acrylic acid and with 2-vinylpyridine.). In some cases, pre-formed polymers can be imprinted to create a MIP for use in a mammal. For example, casting techniques can be utilized to form an imprinted material. In some cases, the preformed polymer can be precipitated in the presence of a template molecule and cross-linked.

A MIP can be formed using any suitable cross-linking method. For example, suitable cross-linkers for polymerization of a MIP can include commercial and custom-made styrenic and methacrylate cross-linkers, derivatives of amino acids, hybrid cross-linkers, bisacrylamides and methacrylamides. For example, a MIP can be formed by free radical vinyl polymerization, electro-polymerization, peroxidase-catalyzed polymerization, carbodiimide-induced polymerization or polycondensation. In some cases, polymerization can occur in aqueous suspension, non-aqueous suspension, or on shell-imprinted core-shell particles. In some cases, polymerization can be accomplished via precipitation, mini-emulsion polymerization, water-in-oil polymerization, or plasma-induced polymerization. In some cases, polymerization can be initiated by thermal methods, photochemical methods, oxidation reactions, or γ-irradiation.

A MIP can rebind a template molecule. In some cases, a target molecule can be the same molecule as a template molecule. In some cases, a target molecule can be a different molecule than a suitable template molecule, but can have a three-dimensional structure or topology similar to a target molecule. In some cases, a target molecule can be a polypeptide and a suitable template molecule can be an epitope of the polypeptide, as recognized by an immune system. In some cases, a target molecule can have similar functional groups as the functional groups present on a suitable template molecule.

Any appropriate assay to assess the affinity of a recognition site of a MIP for a target molecule can be used to determine the binding properties of a MIP. For example, liquid chromatography, high-performance liquid chromatography (HPLC), capillary electro-chromatography, solid-phase extraction, or assays modeled after immunoassays, can be used to determine the target molecule affinity and cross-reactivity of a MIP.

As used herein, the term “capture” refers to binding of a target molecule at a recognition site on a MIP. In some cases, capture can limit the bioavailability of a target molecule in the body of a mammal. For example, a MIP can inactivate a target molecule by binding it in such a way that the target molecule can no longer bind a cell, organelle, DNA, protein, polypeptide or other bioactive molecule, thereby inactivating the target molecule. In some cases, the affinity of a recognition site on a MIP for a target molecule can be at least 10⁴ mol⁻¹ (e.g., at least 10⁵, 10⁶, 10⁷, 10⁸, 10⁹, 10¹⁰, 10¹¹ or 10¹² mol⁻¹). In some cases, a MIP can have several recognition sites capable of binding a target molecule. For example, a MIP can have a binding capacity of at least 10 mg of target molecule per gram of MIP (e.g., at least 20, 30, 40, 50, 60, 70, 80, 90, or 100 mg of target molecule per gram of MIP).

The methods provided herein can be used to capture any appropriate target molecule. For example, a target molecule can be, without limitation, a chemical, a drug, a toxin, an immune complex, a polypeptide, or a disease marker. In some cases, a MIP having a recognition site capable of binding a toxin can be administered to a mammal, under conditions where a toxin is bound to the recognition site on a MIP. A toxin can include drugs, drug metabolites, animal venoms, plant alkaloids, mycotoxins, bacteriotoxins, pesticides, heavy metals, and organic solvents, for example.

For example, a method provided herein can be used to capture excessive drug as a way to treat intoxication. In some cases, a MIP can be prepared to capture an analgesic, an antidepressant, a stimulant, a narcotic, an anesthetic, a sedative, a hypnotic, a cardiovascular drug, a psychedelic, an anxiolytic, or an anticonvulsant. For example, a MIP could be prepared to capture an opioid, such as heroin, in the blood stream or in the cerebrospinal fluid. For example, a MIP can be used to prevent a molecule from entering a cell or binding an opioid receptor, and can be used to prevent respiratory depression and death of a mammal, thereby treating opioid intoxication.

A MIP can be capable of being attracted to a magnetic field. For example, a MIP can contain a material capable of being attracted to a magnetic field. Such materials can include a paramagnetic (e.g., magnesium, molybdenum, lithium, and tantalum), ferromagnetic (e.g., iron, nickel, and cobalt), and superparamagnetic materials (e.g., a particle or nanoparticle). Any type of attachment can be used to attach a MIP and a material capable of being attracted to a magnetic field. For example, a MIP and paramagnetic, ferromagnetic, or superparamagnetic material can be chelated. Examples of MIPs, and MIPs containing material capable of being attracted to a magnetic field, and methods for making such MIPs are described elsewhere (see, e.g., U.S. Pat. No. 6,316,235, U.S. Pat. No. 3,970,518, U.S. Pat. No. 3,985,649, U.S. Pat. No. 4,335,094).

A MIP can be sensitive to a magnetic field source. For example, a MIP with a magnetic particle can be attracted to a device adapted to have a magnetic capture element. In some cases, an alternating magnetic field can be used to produce thermal energy in a MIP with a magnetic particle. For example, an alternating magnetic field can be used to heat a magnetic particle (e.g., the particle temperature can be increased by 10° C.). In some cases, thermal energy can be used to ablate a target, or a cell expressing a target. In some cases, thermal energy can be used to deliver a bound molecule. For example, a thermo-responsive MIP can be used to release a drug in response to an alternating magnetic field.

A MIP can include any appropriate tag. Such tags can include polypeptides (e.g., enzymes or fluorescent reporters), fluorophores (e.g., fluorescent dyes or quantum dots) and contrast agents (e.g., for radiologic imaging, MRI, or PET scanning). For example, a MIP tagged with an enzyme (e.g., a metalloproteinase) can capture an extracellular matrix protein and cleave the captured protein. In some cases, a MIP and cleaved target molecule can be removed from the body of a mammal (e.g., by use of a magnetic field). In some cases, a tagged MIP can cleave a cell from a tissue (e.g., micro-biopsy). In some cases, a tag can be bound to a MIP at a recognition site. For example, a MIP can have multiple recognition sites for binding a tag, and at least one target molecule. For example, an enzyme-tagged MIP can be used to catalyze a reaction upon target substrate binding to a MIP, and capture an enzyme product using the same MIP.

A contrast tagged-MIP can be used to produce an image. For example, a MIP can be used in medical imaging techniques to provide information regarding the presence, and location of a target molecule in the body of a mammal. In some cases, a MIP can be tagged with a radioactive, magnetic, or optical contrast agent. For example, gadolinium-tagged MIP can capture amyloid β on plaques of an Alzheimer's patient, for in vivo imaging of plaque formation.

A MIP can be used to identify a mammal with a pathological condition. For example, a MIP can capture a disease marker. Such markers can indicate the presence of a pathological condition in a mammal. For example, pathological conditions having disease markers can include, without limitation, diabetes, neurodegenerative diseases (e.g., Alzheimer's and Parkinson's diseases), viral infections, carcinomas, cardiovascular diseases, inflammatory diseases, autoimmune diseases. For example, a MIP can capture circulating amyloid β (Aβ40/42), a marker of Alzheimer's disease. In some cases, a MIP can be used to determine whether or not a mammal has a disease. For example, the presence of a MIP-disease marker complex in the body of a mammal can indicate that the mammal has a pathological condition. For example, the presence of a MIP-amyloid β complex in cerebrospinal fluid can be used to identify a human having Alzheimer's disease.

A device such as a guide catheter and a capture element can be used to remove a MIP from the body of a mammal. For example, a guide catheter can be configured to house a capture element and can be configured to be inserted into a blood vessel within a mammal (e.g., a mammal's femoral vein or artery). A capture element can be capable of supplying a magnetic field that can be positioned in the blood stream of a mammal. For example, a method provided herein can use a device configured such that an administered MIP containing a material capable of being attracted to a magnetic field is captured when the mammal's blood flows through the catheter. In some cases, a device can be deployed in the cerebrospinal fluid or in a percutaneous manner. In some cases, a device can be removable from the body of a mammal (e.g., an indwelling catheter) or can be a permanent implant.

A device can include a removal element configured to remove captured items from the mammal in an intermittent or continuous manner. For example, a device can include a removal element that provides suction to a portion of a capture element that captures items (e.g., MIPs). In some cases, suction can draw any captured items from the distal end of the device to a location outside a mammal's body. In some cases, a device can include a dialyzer having a membrane for binding MIPs. For example, a MIP:molecule complex can be removed from the body of a mammal by pumping the mammal's blood through a dialyzer.

The methods herein can be used to capture a percentage (e.g., up to 5, 10, 15, 20, 25, 30, 35, 40, 45, 50, 55, 60, 65, 70, 75, 80, 85, 90, 95, or 100 percent) of the target molecule in a mammal. In some cases, the method provided herein can be used to capture between 5 and 75 percent of the target molecule in a mammal. Such a capture can reduce the bioavailability of a target molecule in a mammal.

The methods herein can be used to capture and remove a percentage (e.g., up to 5, 10, 15, 20, 25, 30, 35, 40, 45, 50, 55, 60, 65, 70, 75, 80, 85, 90, 95, or 100 percent) of the target molecule in a mammal. In some cases, the method provided herein can be used to remove between 5 and 75 percent of the target molecule in a mammal. Such a removal can reduce the amount of target molecule in a mammal.

The methods herein can be used to remove a percentage (e.g., up to 5, 10, 15, 20, 25, 30, 35, 40, 45, 50, 55, 60, 65, 70, 75, 80, 85, 90, 95, or 100 percent) of the MIPs administered to a mammal. In some cases, the method provided herein can be used to remove between 5 and 75 percent of the MIPs administered to a mammal. Such a removal can reduce the amount of the MIPs administered to a mammal.

A MIP can include any appropriate molecule (e.g., a drug) for release into the body of mammal. For example, a MIP can be loaded with a drug to form a MIP-drug complex for administration to a mammal. An appropriate drug can include, without limitation an anti-thrombolytic, anti-neoplastic, anti-parasitic, anti-microbial, or anti-inflammatory drug.

A MIP-molecule complex can have any appropriate dissociation constant (K_{d}) for controlled release of a loaded molecule into the body of a mammal. In some cases, a loaded molecule can have altered bioavailability as compared to the molecule when it is not loaded on a MIP. For example, a loaded molecule could have reduced bioavailability as compared to a molecule that is not loaded on a MIP.

A MIP can be used to deliver a molecule to any appropriate target in the body of a mammal. For example, a MIP can have multiple recognition sites for binding multiple molecules (e.g., a recognition site for binding a molecule to be released and at least one additional recognition site for a target molecule). For example, a MIP-molecule complex can be targeted to any organ, tissue, cell, organelle, or biomolecule (e.g., a nucleic acid or polypeptide) in the body of a mammal, to localize a molecule's pharmacological activity to a site or organ of action. In some cases, a MIP-molecule complex can be targeted to a pathogen (e.g., a virus, a bacterium, a protozoan, a fungus, or a parasite). In some cases, a MIP-molecule complex can target a cell in a cell-type specific manner. For example, a MIP-molecule complex can target a cytotoxic T-cell by binding CD8, or target a breast cancer cell by binding HER-2, and deliver a molecule. In some cases, a MIP-molecule complex can be targeted to a site or organ using a magnetic field. For example, a magnetic MIP-molecule complex can be targeted to a tumor using a localized magnetic field source (e.g., an external magnet).

A MIP can be used to deliver a molecule in response to a stimulus. For example, a MIP-molecule complex can be dissociated upon the application of a stimulus. A stimulus can include an application of a magnetic field, a mechanical deformation, a thermal alteration, optical and magnetic energy transfer, or electrical and chemical signals. For example, a thermo-responsive MIP-drug complex can be dissociated by application of an alternating magnetic field.

Any suitable assay can be used to determine binding of a MIP to a delivery target. For example, a suitable assay can be a displacement assay with a radio-, chromophore-, or fluorophore-linked ligand, a MIP-based precipitation assay using cellular lysate; an enzyme-linked binding assay; or a cytoimmunochemical assay.

A MIP provided herein can be administered to any mammal. For example, a MIP can be administered to a human, a horse, a cow, a goat, a sheep, a dog, a monkey, a cat, a guinea pig, a rat, or a mouse. A MIP provided herein can be administered to any part of a mammal's body. For example, a MIP can be administered to a body cavity, an organ, a body part, or a body fluid. In some cases, a MIP can be administered intravenously, intraarterially, intrathecally, intraabdominally, intramuscularly or subdermally. In some cases, a MIP can be administered to a target organ via a tissue or organ selective blood vessel.

The invention will be further described in the following examples, which do not limit the scope of the invention described in the claims.

### EXAMPLES

### Example 1—Preparation and Characterization of Amyloid-Beta Specific MIPs

A MIP having a recognition site for Amyloid-beta peptide is prepared. A solution of acrylic acid (55 μmol), acrylamide (55 μmol), N-benzylacrylamide (110 μmol), and amyloid-beta (3 μmol) or a polypeptide having an epitope that generated AβP-directed IgM antibody L11.3 or HyL5 (Banks, W. A. et al. Exp Neurol 206(2): 248-256 (2007)) is mixed in a vial of 0.3 mL of acetonitrile water (1:1). The vial is capped, and the mixture is irradiated with UV-light at 350 nm for 6 hours. The polymer, which is formed in the vial, is washed with 20 mM phosphate buffer (pH=3-4) to remove 70 to 80% of the polypeptide template. The buffer-washed polymer is washed with acetonitrile and is dried. After the polymer is dried, it is ground.

The binding characteristics of the amyloid-beta MIPs are determined using an ELISA assay. Amyloid-beta is immobilized on a microtiter plate via capture by L11.3 or HyL5. A control polypeptide is non-specifically adhered to the microtiter plate. After the antigen is immobilized, the MIPs are added. A secondary antibody linked to an enzyme is used to detect a MIP. After the final wash step the plate is developed by adding a fluorogenic substrate to produce a visible signal, which indicates the ability of the MIP to specifically bind a quantity of Amyloid-beta.

### Example 2—Capture and Removal of Amyloid-beta in Mice

Magnetic MIPs specific to Amyloid-beta are prepared as in Example 1 except the protein/monomer mixture includes iron oxide. The ground MIPs are sterilized and suspended in vehicle for intrathecal administration.

The composition of Amyloid-beta specific MIPs is administered to a group of SAMP8 mice via intrathecal injection. The SAMP8 strain of mouse is used as a model for Alzheimer's disease, as it has an age-related overexpression of Aβ that mediates an age-related development of cognitive defects. (Flood & Morley Neurosci Biobehav Rev 22:1-20 (1998)). A composition of control MIPs is administered to a second group of SAMP8 mice.

After 4 hours, a magnetized catheter is inserted into the CSF or peripheral circulation to capture the magnetic MIPS. After 30 minutes, the catheters are removed and the magnetic field reversed to release bound MIPs.

To determine whether the MIPs bound Amyloid-beta, the released MIPs are washed with 20 mM phosphate buffer (pH=3-4). The wash buffer is analyzed for the presence of Amyloid-beta using an ELISA assay.

### Example 3—Targeted Delivery of a VEGF Specific MIP in a Mouse

A composition of magnetic MIPs specific for the polypeptide VEGF is prepared using the epitope that generated an anti-VEGF antibody as in Example 1. The MIPs are sterilized and suspended in a vehicle for interperitoneal administration. Tumors are induced in the right kidneys of nude mice by the injection of cultured Wilms' tumor cells. After one week, anti-VEGF treatment is begun with intraperitoneal injection of either vehicle or a composition of VEGF-specific MIPs. An external magnet is attached to the back of each mouse at the right kidney. Mice are killed after 4 weeks of treatment, and tumor weights and incidences of metastases are evaluated.

### Example 4—Targeted Delivery of a MIP for Thermal Ablation

A composition of magnetic MIPs specific for the polypeptide HER-2 is prepared using the epitope of an anti-HER-2 antibody as in Example 1. The MIPs are sterilized and suspended in a vehicle for subcutaneous administration. Tumors are induced under the skin of nude mice by the injection of cultured HER-2 positive cells. After one week, anti-HER-2 treatment is begun with a subcutaneous administration of vehicle or the composition of HER-2 specific MIPs. Treatment includes heating up the MIPs by about 10° C. by applying an alternating magnetic field. After 4 weeks of treatment, the mice are killed, and tumor weights are evaluated.

### Example 5—Capture and Removal of Amyloid-beta in Humans

Magnetic MIPs specific to Amyloid-beta are prepared as in Example 1 except the protein/monomer mixture includes iron oxide. The ground MIPs are sterilized and suspended in vehicle for intrathecal administration. The objective is to determine the efficacy and safety of administration MIPs and removal of a MIP:Aβ complex in treatment of Alzheimer's disease.

The composition of Amyloid-beta specific MIPs is administered to a group of individuals with mild to moderate Alzheimer's disease (Mini-Mental State Examination scores between 14 and 26, inclusive). After 4 hours, a magnetized catheter is inserted into the CSF or peripheral circulation to capture the magnetic MIPS. After 30 minutes, the catheters are removed and the magnetic field reversed to release bound MIPs. The treatment is repeated monthly, for 6 months. Patients are assessed for a change in the cognitive subscale of the Alzheimer Disease Assessment Scale (ADAS-cog).

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for capturing a molecule present within a mammal, wherein said method comprises administering, to said mammal, a composition comprising a molecularly imprinted polymer comprising a recognition site for said molecule under conditions wherein said molecule binds to said molecularly imprinted polymer to form a molecularly imprinted molecule-molecule complex, thereby capturing said molecule.

2. The method of claim 1, wherein said molecule comprises a disease marker.

3-4. (canceled)

5. The method of claim 1, wherein said molecule comprises a toxin.

6. The method of claim 5, wherein said toxin is a pesticide, a drug, or a heavy metal.

7-12. (canceled)

13. The method of claim 1, wherein said mammal is a human.

14. The method of claim 1, wherein said molecularly imprinted polymer comprises a magnetic particle.

15. The method of claim 1, wherein said molecularly imprinted polymer comprises a contrast agent.

16. The method of claim 1, wherein said molecularly imprinted polymer comprises a polypeptide.

17-20. (canceled)

21. The method of claim 1, wherein said molecule binds to said molecularly imprinted polymer with an affinity of at least 10⁴ mol⁻¹.

22. (canceled)

23. The method of claim 1, wherein said method includes removing said complex.

24. The method of claim 23, wherein said removing step comprises use of a magnetic field.

25. (canceled)

26. A method for releasing a molecule within a mammal, wherein said method comprises administering, to said mammal, a composition comprising a molecularly imprinted polymer and said molecule, wherein said molecule is releasably bound to said molecularly imprinted polymer at a recognition site for binding said molecule to form a molecularly imprinted polymer-molecule complex, under conditions wherein said molecule dissociates from said complex, thereby releasing said molecule.

27. The method of claim 26, wherein said molecule is selected from the group consisting of anti-thrombolytic drugs, anti-neoplastic drugs, and anti-microbial drugs.

28. The method of claim 26, wherein said mammal is a human.

29. The method of claim 26, wherein said molecularly imprinted polymer comprises one or more additional recognition sites.

30. The method of claim 29, wherein one of said one or more additional recognition sites is capable of binding an organ, tissue, or cell.

31. The method of claim 30, wherein said cell is a cancer cell, a microbial cell, or a cell comprising a virus.

32. The method of claim 26, wherein said molecularly imprinted polymer-molecule complex has a dissociation constant of at least 10⁴ mol⁻¹.

33. The method of claim 26, wherein said dissociation condition comprises a stimulus.

34. The method of claim 33, wherein said stimulus comprises a chemical, electrical, magnetic, or mechanical stimulus.
